(19) United States
(12) Patent Application Publication
(10) Pub. No.: US 2022/0144750 A1
(43) Pub. Date: May 12, 2022

(54) DOUBLE ALKOXYCARBONYLATION OF DIENES AS ONE-POT SYNTHESIS

(71) Applicant: EVONIK OPERATIONS GMBH, Essen (DE)

(72) Inventors: Ji YANG, Harbin City (CN); Ralf Jackstell, Rostock (DE); Malthias Beller, Ostseebad Nienhangen (DE); Robert Franke, Marl (DE)

(73) Assignee: EVONIK OPERATIONS GMBH, Essen (DE)

(21) Appl. No.: 17/523,191

(22) Filed: Nov. 10, 2021

(30) Foreign Application Priority Data
Nov. 12, 2020 (EP) 20207181.7

(51) Int. Cl. C07C 67/38

(52) U.S. Cl. CPC C07C 67/38

(57) ABSTRACT

Process for the double alkoxycarbonylation of dienes as one-pot synthesis.

The invention relates to a process for the double alkoxycarbonylation of dienes as one-pot synthesis.

The alkoxycarbonylation of ethylenically unsaturated compounds is a process of increasing significance. An alkoxycarbonylation is understood to mean the reaction of ethylenically unsaturated compounds (olefins) with carbon monoxide and alcohols in the presence of a metal-ligand complex to give the corresponding esters. Typically, the metal used is palladium.

The following scheme shows the general reaction equation for an alkoxycarbonylation:

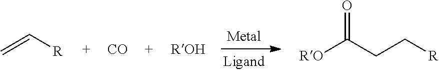

WO 2006/084889 A1 describes a process for preparing a dicarboxylic acid. This comprises the steps of:

(a) contacting a conjugated diene with carbon monoxide and water in the presence of a catalyst system that includes a palladium source, an anion source and a bidentate phosphine ligand to obtain a mixture comprising an ethylenically unsaturated acid and one or more reversible adducts of the conjugated diene and the ethylenically unsaturated acid; and

(b) removing the unreacted conjugated diene and the reversible adducts of the conjugated diene from the reaction mixture; and

(c) further reacting the mixture comprising the ethylenically unsaturated acid obtained in step (b) with carbon monoxide and water to obtain the dicarboxylic acid.

The technical object of the invention is to provide a novel process that, unlike in the prior art mentioned above, does not necessitate the removal from the reaction mixture of unreacted conjugated diene and/or the reversible adducts of the conjugated diene.

The object is achieved by a process according to claim 1.

Process comprising the process steps of:
a) initially charging a diene;
b) adding a ligand of formula (I):

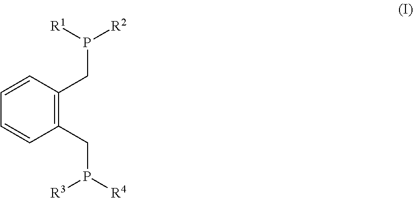

where
R¹, R², R³, R⁴ are selected from: (C₅-C₂₀)-heteroaryl radical, (C₁-C₁₂)-alkyl;
c) adding a compound containing Pd;
d1) adding an alcohol,
   wherein the alcohol is added in an amount at least twice that of the diene, based on the molar ratio;
e) feeding in CO;
f) heating the reaction mixture from a) to e), with conversion of the diene directly into a diester, without at least one of the following components being removed from the reaction mixture:
   unreacted conjugated diene,
   reversible adducts of the conjugated diene.

In the process, step d) comprises process step d1) and optionally further process steps d2).

It is possible here to add the substances in any order. Typically, however, CO is added after the co-reactants have been initially charged in steps a) to d). In addition, CO can also be fed in in two or more steps, in such a way that, for example, a portion of the CO is first fed in, then the mixture is heated, and then a further portion of CO is fed in.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

The expression (C₅-C₂)-heteroaryl encompasses mono- or polycyclic aromatic hydrocarbon radicals having 5 to 20 carbon atoms, where one or more of the carbon atoms are replaced by heteroatoms. Preferred heteroatoms are N, O and S. The (C₅-C₂₀)-heteroaryl groups have 5 to 20, preferably 5 or 6, ring atoms. Thus, for example, pyridyl is in the context of this invention a C₆-heteroaryl radical and furyl is a C₅-heteroaryl radical.

In one variant of the process, at least three of radicals R¹, R², R³, R⁴ are (C₁-C₁₂)-alkyl.

In one variant of the process, at least three of radicals R¹, R², R³, R⁴ are ^{ter}Bu.

In one variant of the process, at least one of radicals R¹, R², R³, R⁴ is a (C₅-C₂₀)-heteroaryl radical.

In one variant of the process, at least one of radicals R¹, R², R³, R⁴ is 2-pyridyl.

In one variant of the process, the (C₃-C₂₀)-heteroaryl radical is 2-pyridyl.

In one variant of the process, the ligand in process step b) has the formula (1):

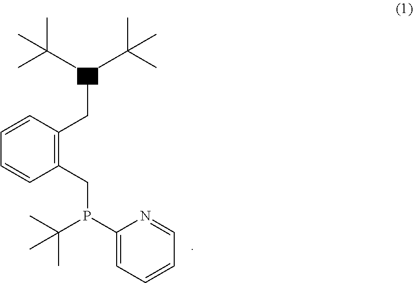

In one variant of the process, the diene in process step a) is selected from: 1,3-butadiene, 1,2-butadiene, vinylcyclohexene.

In one variant of the process, the Pd-containing compound in process step c) is selected from: palladium(II) trifluoroacetate, palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl)dichloride, palladium iodide, palladium diiodide.

Preferably, the Pd-containing compound is Pd(TFA)₂, Pd(dba)₂, Pd(acac)₂ or Pd(OAc)₂.

Particularly suitable are Pd(TFA)₂ and Pd(acac)₂.

The molar ratio of Pd:ligand is preferably in the range from 1:1 to 1:10, preferably from 1:1 to 1:6, more preferably from 1:1 to 1:4.

In one variant of the process, the alcohol in process step d1) is selected from: methanol, ethanol, 1-propanol, 1-butanol, 1-pentanol, 1-hexanol, 2-propanol, tert-butanol, 3-pentanol.

In one variant of the process, the alcohol in process step d1) is methanol.

In one variant of the process, the process comprises the additional process step d2) of:
d2) adding a Brønsted acid.

In one variant of the process, the Brønsted acid in process step d2) is para-toluenesulfonic acid.

In one variant of the process, in process step f) the component:
unreacted conjugated diene,
   is not removed from the reaction mixture.

In one variant of the process, in process step f) the component:
reversible adducts of the conjugated diene,
   is not removed from the reaction mixture.

In one variant of the process, in process step f) the following two components:
unreacted conjugated diene,
reversible adducts of the conjugated diene,
   are not removed from the reaction mixture.

In one variant of the process an organic solvent that is not an alcohol is added to the reaction mixture. This is preferably selected from: toluene, xylene, anisole, chlorobenzene, THF, methylfuran, propylene carbonate, cyclohexane, alkane, ester, ether. Particular preference is given to toluene.

CO is in process step e) fed in preferably at a CO partial pressure in the range from 0.1 to 10 MPa (1 to 100 bar), preferably from 1 to 5 MPa (10 to 50 bar), more preferably from 3 to 5 MPa (30 to 50 bar).

In one variant of the process, the reaction mixture is heated in process step f) of the process according to the invention to a temperature in the range from 30° C. to 150° C., preferably from 40° C. to 140° C., more preferably from 60° C. to 130° C., in order to convert the ethylenically unsaturated compound into a diester.

The invention is to be illustrated in detail hereinafter by a working example.

Experimental Procedures

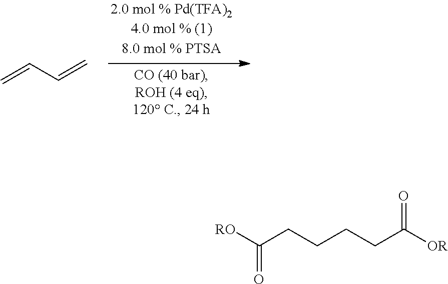

In general, an excess of ligand was used in all catalyst experiments in order to ensure the stability of the active complex at low metal concentration. All experiments used 1,3-butadiene as gas. The butadiene was condensed at low temperature into a special metal tube (this metal tube may be connected to the corresponding autoclave) and the corresponding mass of butadiene was accurately weighed to determine the amount added to the autoclave. A 100 ml autoclave was used. First, the autoclave was evacuated and then filled with argon (3× repetition of this process). The solids were then added to the autoclave under an argon atmosphere. Four equivalents of methanol (based on the amount of butadiene) and toluene were used. The volume ratio of methanol and toluene was varied in the series of experiments. The reactions were stirred for 24 hours at the specified temperature. The autoclave was then cooled to room temperature and the pressure cautiously released. Mesitylene (0.5 mmol) was added to the reaction as internal standard. A sample of the mixture was analysed by gas chromatography. Pure product was obtained by column chromatography on silica gel (eluent: pentane/ethyl acetate=40).

Reaction Conditions

Alcohol: MeOH/ⁿBuOH (4 equiv. based on the olefin)

Pressure (CO): 40 bar

Temperature: 120° C.

Reaction time: 24 h

Ligand: (1)

Pd:ligand=2 mol %:4 mol % (based on the olefin)

Experimental Results

| Alcohol | Yield of diester [%] |
|---|---|
| MeOH | 86 |
| ⁿBuOH | 85 |

1. Process comprising the process steps of:
a) initially charging a diene;
b) adding a ligand of formula (I): 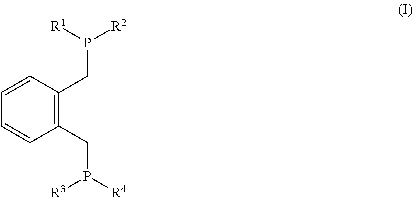
where
R¹, R², R³, R⁴ are selected from: (C₅-C₂₀)-heteroaryl radical, (C₁-C₁₂)-alkyl;
c) adding a compound containing Pd;
d1) adding an alcohol,
wherein the alcohol is added in an amount at least twice that of the diene, based on the molar ratio;
e) feeding in CO;
f) heating the reaction mixture from a) to e), with conversion of the diene directly into a diester, without at least one of the following components being removed from the reaction mixture:
unreacted conjugated diene,
reversible adducts of the conjugated diene.

2. Process according to claim 1,
wherein at least three of radicals R¹, R², R³, R⁴ are (C₁-C₁₂)-alkyl.

3. Process according to claim 1,
wherein at least three of radicals R¹, R², R³, R⁴ are ^{ter}Bu.

4. Process according to claim 1,
wherein at least one of radicals R¹, R², R³, R⁴ is a (C₅-C₂₀)-heteroaryl radical.

5. Process according to claim 1,
wherein at least one of radicals R¹, R², R³, R⁴ is 2-pyridyl.

6. Process according to claim 1,
wherein the (C₃-C₂₀)-heteroaryl radical is 2-pyridyl.

7. Process according to claim 1,
wherein the ligand in process step b) has the formula (1):

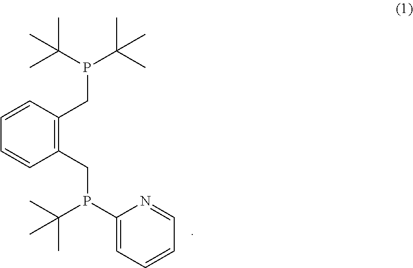 [SMILES: CC(C)(C)P(Cc1ccccc1CP(C(C)(C)C)C(C)(C)C)c1ccccn1]

8. Process according to claim 1,
wherein the diene in process step a) is selected from: 1,3-butadiene, 1,2-butadiene, vinylcyclohexene.

9. Process according to claim 1,
wherein the Pd-containing compound in process step c) is selected from: palladium(II) trifluoroacetate, palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), palladium(cinnamyl)dichloride, palladium iodide, palladium diiodide.

10. Process according to claim 1,
wherein the alcohol in process step d1) is selected from: methanol, ethanol, 1-propanol, butanol, 1-pentanol, 1-hexanol, 2-propanol, tert-butanol, 3-pentanol.

11. Process according to claim 1,
wherein the process comprises the additional process step d2) of:
d2) adding a Brønsted acid.

12. Process according to claim 1,
wherein in process step f) the component:
unreacted conjugated diene,
is not removed from the reaction mixture.

13. Process according to claim 1,
wherein in process step f) the component:
reversible adducts of the conjugated diene,
is not removed from the reaction mixture.

14. Process according to claim 1,
wherein in process step f) the following two components:
unreacted conjugated diene,
reversible adducts of the conjugated diene,
are not removed from the reaction mixture.
